(19) United States
(12) Patent Application Publication
(10) Pub. No.: US 2026/0179766 A1
(43) Pub. Date: Jun. 25, 2026

(54) HALO-SAFE AI AND METHODS OF MAKING AND USING SAME

(71) Applicants: Mark T. McBride, Coronado, CA (US); John Roughneen, Dennis, MA (US); Glenn Butler, Holliston, MA (US)

(72) Inventors: Mark T. McBride, Coronado, CA (US); John Roughneen, Dennis, MA (US); Glenn Butler, Holliston, MA (US)

(21) Appl. No.: 19/398,910

(22) Filed: Nov. 24, 2025

Related U.S. Application Data
(60) Provisional application No. 63/736,987, filed on Dec. 20, 2024.

(51) Int. Cl. G16H 40/40; A41D 13/002; A41D 13/12; G16H 40/63

(52) U.S. Cl. CPC G16H 40/40; A41D 13/0025; A41D 13/1218; G16H 40/63

(57) ABSTRACT

Systems, methods, and other embodiments for a novel analyzing operating condition parameters for medical devices, and more specifically, to a system and method that can analyze operating condition parameters of medical devices and determine if there is a correlation between the various operating condition parameters of a medical device and how the medical device is operating and provide (or predict) a recommended corrective action to correct the operating conditions of the medical device.

### CROSS-REFERENCE TO RELATED APPLICATION

This application claims benefit of U.S. Patent Application 63/736,987, filed on Dec. 20, 2024, the disclosure of which is hereby incorporated by reference in its entirety to provide continuity of disclosure to the extent such a disclosure is not inconsistent with the disclosure herein.

### FIELD OF THE INVENTION

The present invention generally relates to system and method for analyzing operating condition parameters for medical devices, and more specifically, to a system and method that can analyze operating condition parameters of medical devices and determine if there is a correlation between the various operating condition parameters of a medical device and how the medical device is operating and provide (or predict) a recommended corrective action to correct the operating conditions of the medical device.

### BACKGROUND OF THE INVENTION

Prior to the present invention, as set forth in general terms above and more specifically below, it is known that surgical personnel can use medical devices during surgical procedures that are equipped with monitors. These monitors can be equipped to keep track of operating condition parameters (or air quality) within a medical device such as a surgical hood, wherein the operating condition parameters include, but are not limited to, carbon dioxide (CO₂), temperature, humidity, oxygen (O₂), volatile organic compounds (VOCs), and/or air pressure. It is also known that one-piece surgical gowns are designed to cover the wearer completely and sterilely when they are attached to the hood. Currently, a helmet or other similar head support structure is donned by the wearer and the one-piece gown and the hood are conventionally attached to the helmet or other similar head support structure. Furthermore, it is known to provide a ventilation system within the helmet that is also attached to the helmet or other similar head support structure or attached to the wearer.

When the hood and gown are donned by the wearer, it is important that the hood and gown completely and sterilely cover the wearer, as discussed above. In this manner, a closed area is created around the wearer's head and neck areas. Ventilation must be provided within this closed area so that the wearer can don the hood and gown and still be able to properly perform the medical procedure without having to worry about encountering high levels of carbon dioxide (CO₂), temperature, humidity, oxygen (O₂), VOCs. and/or air pressure within the closed area.

Furthermore, it is known that medical devices need to be properly installed and maintained to function properly. A medical device that is not properly installed or maintained may fail at a very inopportune time, which could lead to a serious incident during a medical operation or procedure. For example, if the medical device is not properly installed or maintained, the wearer may experience high levels of carbon dioxide (CO₂), temperature, humidity, oxygen (O₂), VOCs, and/or air pressure within the closed area.

While it is advantageous to be able to monitor the operating conditions of the medical device during medical procedures, it would be desirable to be able to determine if one or more of the operating conditions exceeds an operating condition parameter or range, and to determine if these operating conditions that exceed an operating condition parameter or range would adversely affect the operation of the medical device. For example, assume that a surgeon is performing a surgical procedure, and the surgeon is wearing a surgical hood that includes a ventilation system equipped with a fan. Also, assume that the monitoring system that is keeping track of the operating condition parameters of the fan determines that the CO₂ levels, the humidity levels, and the air pressure within the surgical hood are exceeding their operating condition parameter levels.

In this instance, it would be desirable if the system and method were able to perform an analysis of the operating condition parameters analysis for the fan. It would be even further advantageous if the system and method can analyze operating condition parameters of medical devices and determine if there is a correlation between the various operating condition parameters of a medical device and how the medical device is operating. For example, the system and method could be trained to review previous instances where a similar fan encountered similar operating conditions, and it was determined by the system and method that these similar operating conditions correlated with the replacing of the air filter in the fan in order to correct the abnormal operating conditions. In this manner, the system and method could provide feedback to the user or system administrator regarding the operating condition parameters of the system while the system is being used and provide recommended ways to correct the abnormal operating conditions of the medical device. Finally, the system and method would be able to collect information about the operating parameter conditions of the medical devices for user edification and/or further product development.

It is a purpose of this invention to fulfill these and other needs in the medical device art in a manner more apparent to the skilled artisan once given the following disclosure.

The preferred system and method for managing operating condition parameters in medical devices, according to various embodiments of the present invention, offers the following advantages: ease of use; the ability to keep track of operating condition parameters in medical devices; the ability to train the system and method to be able to correlate various operating condition parameters with previous recommendations on how to correct the operating condition parameter in the medical device; the ability to the ability to provided recommendations on how to correct the operating condition parameter in the medical device without user intervention; and the ability to provide feedback regarding the operating condition parameters in the medical device for wearer edification, preventative maintenance, and/or further product development. In fact, in many of the preferred embodiments, these advantages are optimized to an extent that is considerably higher than heretofore achieved in prior, known systems and methods for managing operating condition parameters in medical devices.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, isometric view of a helmetless support for use with surgical hoods and gowns, according to one embodiment of the present invention;

FIG. 2 is a schematic, isometric view of a ventilation system for use with surgical hoods and gowns, constructed according to an embodiment of the present invention;

FIG. 3 is a schematic, side view of the ventilation system for use with surgical hoods and gowns illustrating the adjustable face vent air flow levers, constructed according to an embodiment of the present invention;

FIG. 4 is a cut-away view, taken along lines 4-4 of FIG. 3 of the ventilation system for use with surgical hoods and gowns illustrating the adjustable face vent air flow levers, constructed according to an embodiment of the present invention;

FIG. 5 is a schematic, isometric, back view of the ventilation system for use with surgical hoods and gowns with the back cover removed illustrating the power module, constructed according to an embodiment of the present invention;

FIG. 6 is an isometric, back view of the ventilation system for use with surgical hoods and gowns with the back cover removed illustrating the printed circuit board (PCB) module, constructed according to an embodiment of the present invention;

FIG. 7 is a schematic illustration of the ventilation system for use with surgical hoods and gowns illustrating the operating condition parameter measurement device being located adjacent to the wearer in one embodiment, constructed according to an embodiment of the present invention;

FIG. 8 illustrates one embodiment for a method for managing operating parameters in medical devices;

FIG. 9 is a schematic illustration of a system for managing operating parameters in medical devices, according to the present invention;

FIG. 10 is a schematic illustration of a cloud computing system, according to the present invention;

FIG. 11 illustrates one embodiment for a method for analyzing operating condition parameters analysis for medical devices, according to the present invention;

FIG. 12 illustrates one embodiment for training a predictive model with a collected set of results, according to the present invention;

FIG. 13 illustrates one embodiment for determining if there is a correlation between the various operating condition parameters of a medical device and how the medical device is operating and provide a recommended corrective action to correct the operating conditions of the medical device, according to the present invention; and

FIG. 14 illustrates an embodiment of a computing system configured with the example systems and/or methods disclosed.

### DETAILED DESCRIPTION OF THE PREFERRED EMBODIMENTS OF THE INVENTION

In order to address the shortcomings of the prior, known systems and methods for managing operating parameters in a medical device, it would be desirable to be able to determine if one or more of the operating conditions exceeds an operating condition parameter or range, and to determine if these operating conditions that exceed an operating condition parameter or range would adversely affect the operation of the medical device. For example, assume that a surgeon is performing a surgical procedure, and the surgeon is wearing a surgical hood that includes a ventilation system equipped with a fan. Also, assume that the monitoring system that is keeping track of the operating condition parameters of the fan determines that the CO₂ levels, the humidity levels, and the air pressure within the surgical hood are exceeding their operating condition parameter levels.

In this instance, it would be desirable if the system and method were able to perform an analysis of the operating condition parameters analysis for the fan. It would be even further advantageous if the system and method can analyze operating condition parameters of medical devices and determine if there is a correlation between the various operating condition parameters of a medical device and how the medical device is operating. For example, the system and method could be trained to review previous instances where a similar fan encountered similar operating conditions, and it was determined by the system and method that these similar operating conditions correlated with the replacing of the air filter in the fan in order to correct the abnormal operating conditions. In this manner, the system and method could provide feedback to the user or system administrator regarding the operating condition parameters of the system while the system is being used and provide recommended ways to correct the abnormal operating conditions of the medical device. Finally, the system and method would be able to collect information about the operating parameter conditions of the medical devices for user edification and/or product improvement.

A unique aspect of the present invention is that the system and method can be configured to monitor and correlate the following operating condition parameters of a medical device:
1. Fan speed input in cubic feet per minute (cfm)
2. Revolutions per minute (RPM) of the fan
3. Air Filters and filter life expectancy
4. CO₂ and O₂ levels
5. Air pressure levels
6. Temperature within the surgical hood and the operating room
7. Humidity within the surgical hood and the operating room
8. Volatile organic compound levels (VOCs)
9. Time since surgery started
10. Battery State of Charge (SOC)
11. Light brightness of any light being used within the surgical hood

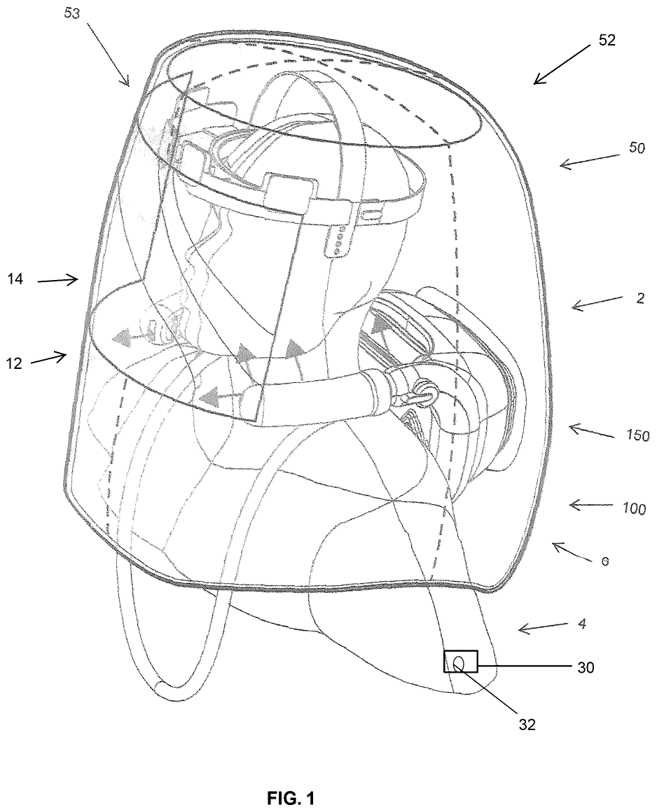

Referring now to FIG. 1-7, there is illustrated a helmetless support system 2 for use with surgical hoods and gowns. The helmetless support system 2 for use with surgical hoods and gowns can be used to support the one-piece surgical gown 4 and the surgical hood 14 without the need for the wearer 6 to wear a helmet. In this manner, one-piece surgical gown 4 and the surgical hood 14 completely and sterilely covers the head, neck, and torso of the wearer 6 when donned by the wearer 6. Also, the one-piece surgical gown 4 and the surgical hood 14 includes a clear faceplate 12. The helmetless support 2 further includes a helmetless surgical hood and gown support having a flexible headband 52 with attached lightweight front offsets 53 in front that can be releasably attached to the faceplate 12. Furthermore, the front offsets 53 are used to provide air circulation around head of the wearer 6.

### Helmetless Surgical Hood and Gown Support

As shown in FIG. 1, helmetless support 2 for use with surgical hoods and gowns includes, in part, surgical gown 4, wearer 6, gown wireless identification system 30, and helmetless surgical hood and gown support 50. It is to be understood that surgical gown 4 is constructed of any suitable, durable, medical grade material. It is to be further understood that the surgical gown 4 is to be constructed into a one-piece design that will completely and sterilely cover the wearer when attached to the hood 14. Finally, it is to be understood that gown wireless identification system 30 is a conventional wireless identification system having an RFID tag 32 that can be conventionally attached to the surgical gown 4.

With respect to helmetless surgical hood and gown support 50, helmetless surgical hood and gown support 50 includes, in part, flexible, adjustable band 52 and front offsets 53. Preferably, flexible band 52 is constructed of any suitable, durable, flexible, medical grade material. An important feature of flexible band 52 being that it comfortably fits around the head of the wearer 6, but still is capable of securely holding surgical gown 4 and surgical hood 14 once the surgical gown 4 and surgical hood 14 have been attached to helmetless surgical hood and gown support 50 and then placed over the wearer, as will be discussed in greater detail later. In particular, it is important that flexible band 52 be able to securely hold hood 14 away from the head of wearer 6 and allow the air to flow around the head of wearer 6, as will be discussed in greater detail later.

### Helmetless Surgical Hood and Gown Ventilation System

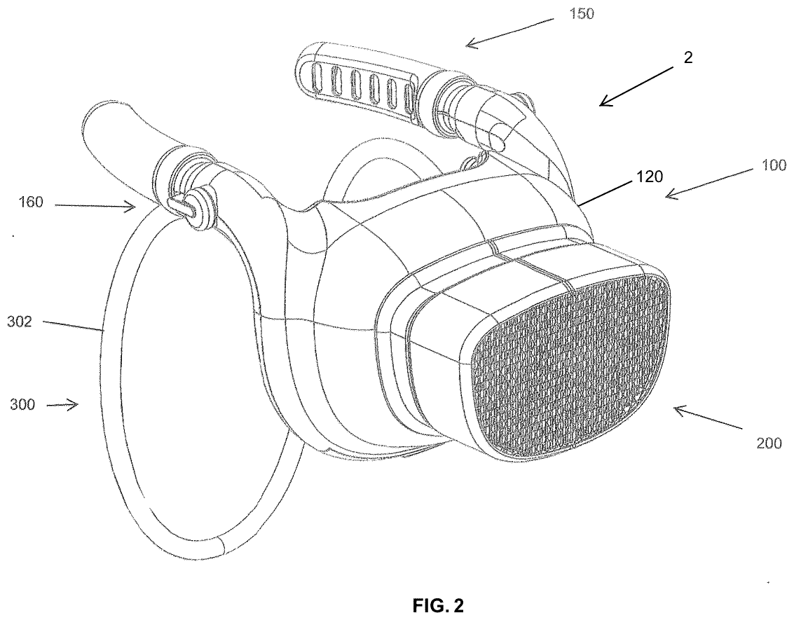

Referring now to FIGS. 2-7, there is illustrated a ventilation system 100 for use with helmetless support system 2. The ventilation system 100 is constructed such that the fan speed can be controlled by the wearer 6 and/or the system 2 once the gown 4 and hood 14 have been donned. A face vent module 150 is used as a “yoke” to support the ventilation system 100 on the shoulders of the wearer 6 (FIG. 1). Finally, the wearer 6 can control the output from each of the various output apertures in face vent module 150 and neck vent module 350 in the ventilation system 100, as will be discussed in greater detail later.

As shown in FIGS. 2-7, helmetless support system 2 for use with surgical hoods and gowns having ventilation system 100 includes, in part, protective casing 120, face vent module 150, air filtration module 200, power module 250, yoke module 300, neck vent module 350, air flow generation module 450, printed circuit board (PCB) module 500, and the operating parameter measurement assembly 600.

A unique aspect of the present invention is the location of the ventilation system 100 with respect to the surgical gown 4 and surgical hood 14. As shown in FIG. 1, the ventilation system 100 is almost completely located inside of the surgical gown 4 and surgical hood 14.

### Protective Casing 120

With respect to protective casing 120, protective casing 120, preferably, is constructed of any suitable, durable, high strength, shock resistant, UV resistant, medical grade polymeric material. It is to be understood that protective casing 120 is used to encase ventilation system 100 to provide protection for air filtration module 200, power module 250, neck vent module 350, air flow generation module 450, and printed circuit board (PCB) module 500.

### Face Vent Module 150

Regarding face vent module 150, as shown in FIGS. 2-6, face vent module 150, includes, in part, removable face vents 152, face vent openings 154, face vent connectors 156, face vent adaptors 158, face vent air flow adjustors 160, and face vent air flow adjuster lever 162. Preferably, face vents 152 and face vent connectors 156 are constructed as a single-piece construction and are constructed of any suitable, durable, lightweight, medical grade, and washable material. Also, face vent openings 154 are formed in removable face vents 152 by conventional techniques such as forming, stamping, molding, or the like. Face vent adaptors 158, preferably, are constructed of any suitable, durable, high strength, medical grade material and are permanently connected to protective casing 120 near face vent air flow adjustors 160 and face vent air flow adjuster levers 162. Finally, face vent air flow adjustors 160 and face vent air flow adjuster lever 162, preferably, are constructed of any suitable, durable, high strength, medical grade material.

A unique aspect of the present invention is the use of removable face vents 152. In particular, removable face vents 152 are constructed in such a manner that allows the removable face vents 152 to be easily removed from the face vent adaptors 158 so that the removable face vents 152 can be cleaned, disinfected, and sanitized prior to the next usage of the helmetless support 2 for use with surgical hoods and gowns having ventilation system 100. Once the removable face vents 152 have been cleaned, disinfected, and sanitized, the removable face vents 152 can be easily slid onto the face vent adaptors 158 by locating the face vent connectors 156 on the face vent adaptors 158.

A further unique aspect of the present invention is the use of face vent air flow adjustors 160 and face vent air flow adjuster lever 162. In particular, the wearer 6 can adjust the amount of air flow that is being emitted out of the removable face vents 152 through the use of vent air flow adjustor 160 and face vent air flow adjuster lever 162. In this manner, the wearer 6 can conventionally manipulate face vent air flow adjuster lever 162 so that the amount of air flow is adjusted. For example, the wearer 6 may push/pull the face vent air flow adjuster lever 162 upwards which will cause the amount of air flow being emitted out of the removable face vents 152 to be reduced. Conversely, the wearer 6 may push/pull the face vent air flow adjuster lever 162 downwards which will cause the amount of air flow being emitted out of the removable face vents 152 to be increased.

### Air Filtration Module 200

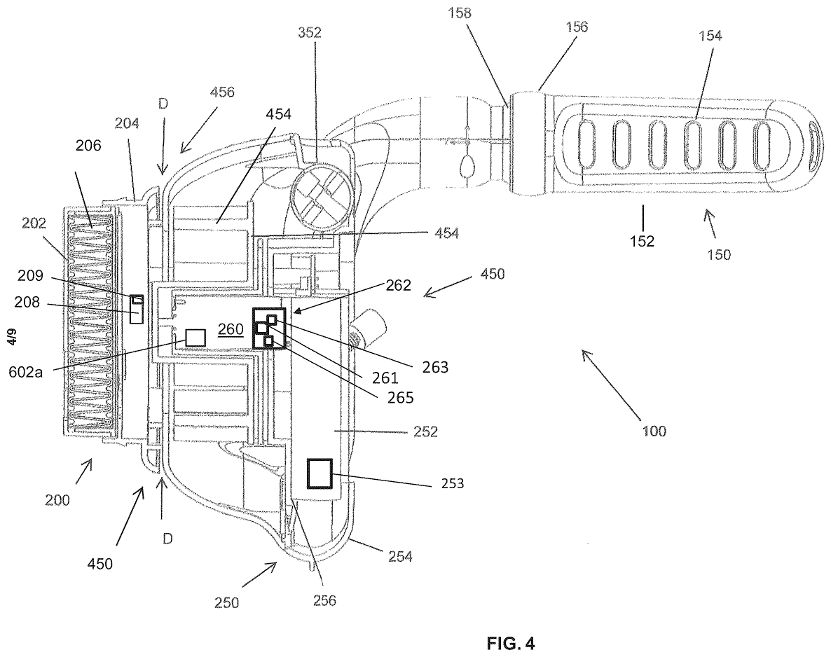

With respect to air filtration module 200, as shown in FIG. 4, air filtration module 200, includes, in part, air filter 202, air filtration adaptor 204, filter casing 206, and air filtration module wireless identification system 208. Preferably, air filter 202 is a HEPA (or ULPA) air filter that is located within filter casing 206. Preferably, filter casing 206 is constructed of any suitable, durable, high strength, medical grade material. Preferably, air filtration adaptor 204 is conventionally formed on protective casing 120. Finally, it is to be understood that air filtration module wireless identification system 208 is a conventional wireless identification system having an RFID tag 209 that can be conventionally attached to or electrically connected to the air filter 202, as will be discussed in greater detail later.

A unique aspect of the present invention is the use of air filtration module 200. In particular, air filtration module 200 can be used to filter out air borne contaminants so that they do enter into the surgical hood 14 and surgical gown 4. As discussed above, only air filter 202 extends outside of the surgical hood 14 (FIG. 1). In this manner, only air going through the air filtration module 200 will be allowed to enter into the surgical hood 14 and surgical gown 4. Also, the air filter 202 can be easily removed and replaced. For example, wearer 6 can simply remove the air filter 202 and the filter casing 206 from the air filtration adaptor 204. The wearer 6 can then replace the used air filter 202 and filter casing 206 with a new air filter 202 and filter casing 206 by simply placing the new air filter 202 and filter casing 206 onto the air filtration adaptor 204. It is to be understood that the air filter 202 and filter casing 206 can be retained on the air filtration adaptor 204 by a snap fit, a threaded connection, a bayonet connection, a slidable connection or the like, as will be discussed in greater detail later.

### Power Module 250

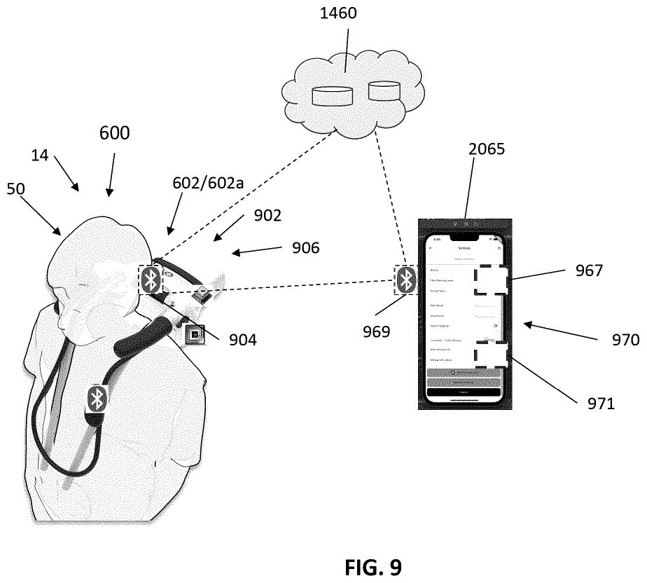

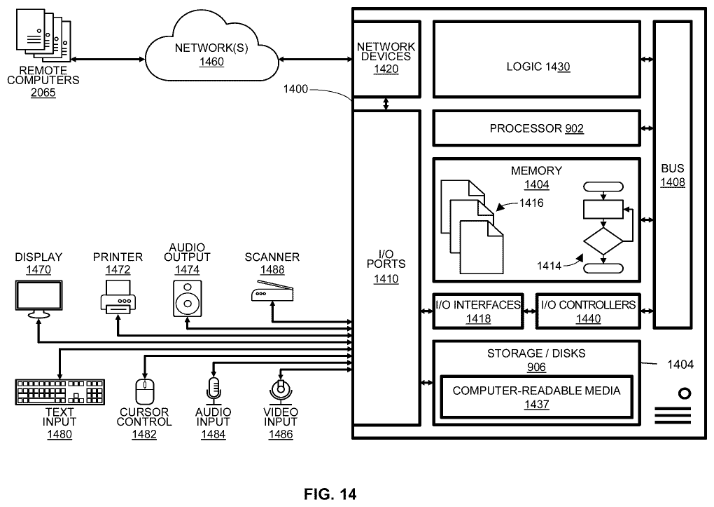

Regarding power module 250, as shown in FIGS. 4-6, power module 250, includes, in part, battery 252, battery sensor 253, battery doors 254, battery lock 256, wireless power module identification system 258, motor sensor/wireless identification system 262, tachometer 263, and a printed control board 265. Preferably, battery 252 is a conventional, rechargeable battery such as a lithium-ion battery or the like that is capable of providing sufficient power to air flow generation module 450, printed circuit board (PCB) module 500, and operating condition parameter measurement assembly 600 for an extended period of time such as 6-8 hours. Battery sensor 253 is a conventional sensor that is configured to be used to monitor the operating status of battery 252 to ensure that battery 252 is operating properly. It is to be understood that the battery sensor 253 is configured to continuously send data to the printed control board 265 (and processor 902) regarding the operating status of the battery 252. Printed control board 265 is configured to send the data to a conventional printed circuit board 502 and the printed circuit board 502 is configured to transmit this information to processor 902. Processor 902 is configured to store this information on storage such as storage 906 (FIG. 14). Also, battery doors 254, preferably are constructed of any suitable, durable, high strength, medical grade material. It is to be understood that wireless power module identification system 258 is a conventional wireless identification system such as an RFID tag that can be conventionally attached to or electrically connected to the battery 252, as will be discussed in greater detail later. It is to be understood that instead of an RFID tag, the battery 252 may include a serial number that can be conventionally read/detected using a hardware data line that is configured to be used with the battery 252 through the wireless power module identification system 258. It is to be further understood that motor sensor/wireless identification system 262 includes a conventional printed circuit board 265, a tachometer 263, a RFID tag 261 that can be conventionally attached to or electronically connected to the fan motor 260, as will be discussed in greater detail later. Furthermore, printed circuit board 265 is configured with an algorithm that is used to determine the operating speed of the fan motor 260 based on one or more of the previously discussed pre-determined thresholds. In particular, the tachometer 263 is configured to monitor the operating speed of the fan motor 260 and continuously send data to the printed control board 265 (and processor 902) regarding the operating speed of the fan motor 260. Printed control board 265 is configured to send the data to a conventional printed circuit board 502. Printed circuit board 502 is configured to transmit this information to processor 902. Processor 902 is configured to store this information on storage such as storage 906 (FIG. 14). Finally, in one embodiment, the algorithm could be updated with information uploaded onto the printed circuit board 265 through an interaction with processor 902 (FIG. 9) either manually or automatically based upon the desired interval in which such information is to be uploaded onto the printed circuit board 265.

A unique aspect of the present invention is the use of battery sensor 253. In particular, battery sensor 253 can be used to monitor the cycles and voltages of battery 250 so that it can be determined when the battery 250 needs to be replaced. Also, the battery sensor 253 can be used in conjunction with the processor 902 (FIG. 9) and printed circuit board (PCB) module 500 to utilize the information from battery sensor 253 in order to alert the user that the battery 250 is not functioning properly or to simply place an order for a replacement for battery 250.

Another unique aspect of the present invention is the use of battery doors 254. Battery doors 254 are conventionally connected to protective casing 120 so that battery doors 254 can swing (or pivot) open so that battery 252 can be easily installed into power module 250 or removed from power module 250. In particular, the wearer 6 can remove battery 252 from power module 250 by opening battery doors 254 and removing battery 252 from power module 250. The battery 252 can then be placed on a conventional battery charger (not shown). Once battery 252 has been fully charged, the wearer 6 can then remove the battery 252 from the battery charger, open the battery doors 254, and slide the battery 252 into the power module 250 so that the battery 252 is securely retained within the power module 250. The wearer 6 then closes the battery doors 254 so that the battery 252 is not exposed to the elements. It is to be understood that a conventional locking mechanism 256 can be used to lock the battery 252 in place in the power module 250 so that the battery 252 does not inadvertently come loose while the ventilation system 100 is being operated.

### Yoke Module 300

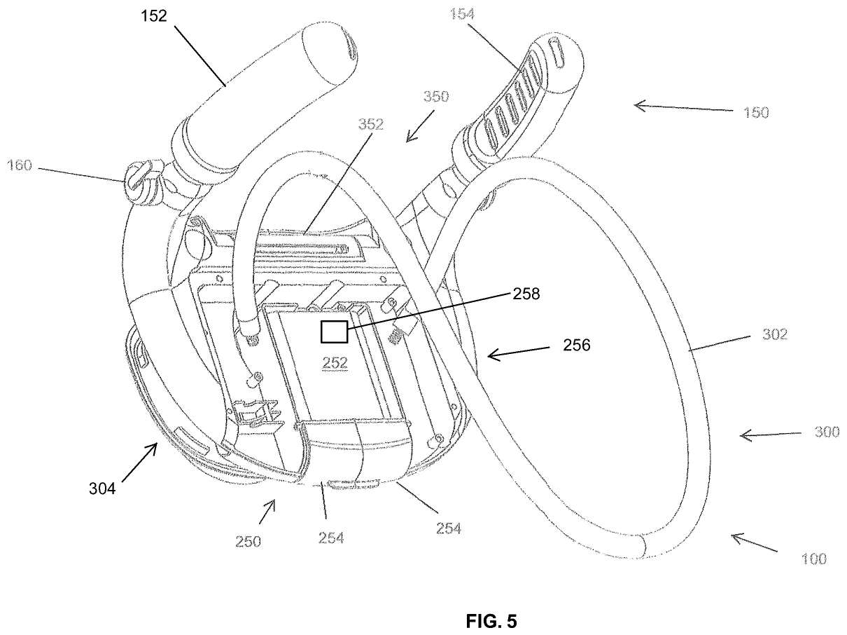

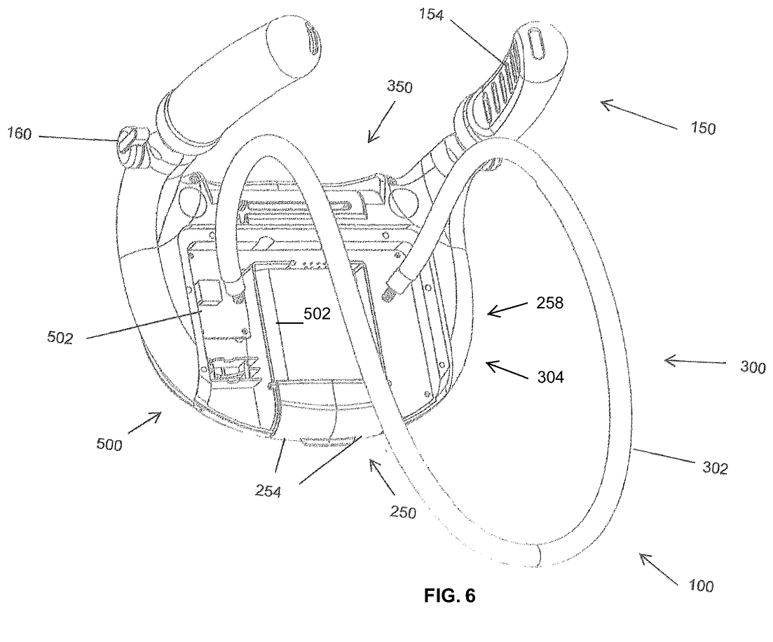

With respect to yoke module 300, as shown in FIGS. 5 and 6, yoke module 300, includes, in part, yoke 302 and yoke connectors 304. Preferably, yoke 302 is constructed of any suitable, durable, high strength, flexible, medical grade material. Preferably, yoke connectors 304 are attached to the back of protective casing 120.

Another unique aspect of the present invention is the use of yoke module 300. In particular, yoke module 300 can be used to assist in retaining ventilation system 100 on the shoulders of the wearer 6. Furthermore, yoke 302 is removably attached to protective casing through the use of yoke connectors 304. In this manner, yoke 302 can be easily attached to and removed from protective casing 120. Furthermore, since yoke 302 is flexible, yoke 302 can be adjusted to fit the upper torso of the wearer 6 so that ventilation system 100 will remain securely retained on the shoulders and the upper torso of the wearer 6. For example, wearer 6 can position the ventilation system 100 with the yoke module 300 installed over his/her head and place the yoke module 300 on the upper torso of the wearer 6 (FIG. 1). The wearer 6 can then pull/push on yoke 302 while yoke 302 is connected to yoke connectors 304 so that yoke 302 firmly contacts the upper torso of the wearer 6 to assist in retaining the ventilation system 100 on the shoulders and upper torso of the wearer 6.

### Neck Vent Module 350

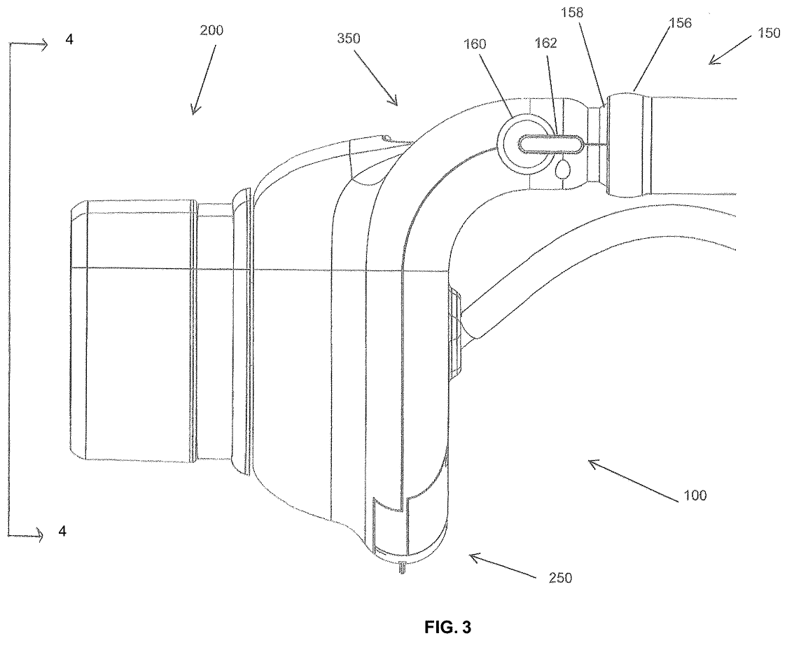

Regarding neck vent module 350, as shown in FIGS. 3-6, neck vent module 350 includes, in part, neck vent 352 (FIG. 4). Preferably, neck vent 352 is constructed of any suitable, durable, high strength, medical grade material.

### Air Flow Generation Module 450

Regarding air flow generation module 450, as shown in FIG. 4, air flow generation module 450 includes, in part, conventional fan motor 260, conventional impeller 454, and back flow opening 456. It is to be understood that battery 252 provides the electrical power to fan motor 260.

Another unique aspect of the present invention is the use of air flow generation module 450. In particular, as the fan motor 260 causes the impeller (or fan) 454 to rotate, the configuration of the impeller 454 causes air to be drawn through the air filter module 200. In this manner, the air filter module 200 can be used to filter the air being drawn into the ventilation system 100. Also, the back flow opening 456 is provided to allow air that is contained within the surgical hood 14 to also be drawn through back flow opening 456 in the direction of arrow D. In this manner, the back flow opening 456 provides for an even greater circulation of the air within the hood 14 while the ventilation system 100 is in operation.

### Printed Circuit Board (PCB) Module 500

With respect to printed circuit board (PCB) module 500, as shown in FIG. 6, printed circuit board module 500, includes, in part, a conventional printed circuit board 502. It is to be understood that processor 902 and printed circuit board 502 are configured to be able to control the ventilation system 100 and interact with gown wireless identification system 30, air filtration module wireless identification system 208, power module wireless identification system 258, motor sensor/wireless identification system 262, and operating condition parameter measurement system 600, as will be described in greater detail later. In particular, processor 902 and printed circuit board 502 can be configured to control the speed at which the impeller 454 (FIG. 4) rotates, thereby controlling the velocity of the air being emitted from the face vents 152 and the neck vent 352. It is to be further understood that the printed circuit board 502 is located in the rear of the protective casing 120 so that the printed circuit board 502 can be located adjacent to battery 252. Finally, it is to be understood that the printed circuit board 502 is conventionally retained within the protective casing 102 by conventional fasteners (not shown). Finally, it is to be understood that the printed circuit board (PCB) module 500 can utilize Bluetooth® low energy capabilities in order to allow the printed circuit board (PCB) module 500 to communicate with a mobile application 967 that is conventionally installed on a remote computer 2065 (i.e., mobile communication device) (FIG. 9) such as a smartphone, tablet, or data collection point.

### Operating Condition Parameter Measurement Assembly 600

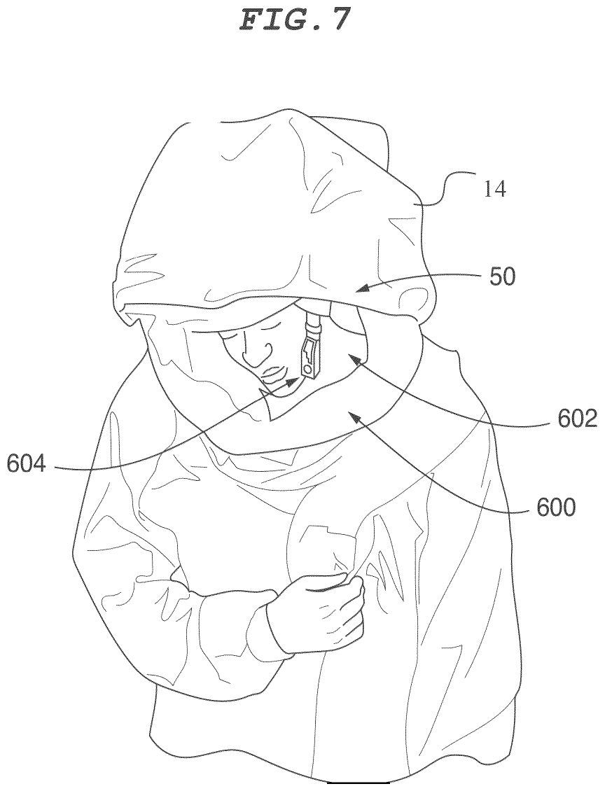

Regarding operating condition parameter measurement assembly 600, as shown in FIGS. 4 and 7, operating condition parameter measurement assembly 600, includes, in part, hood 14, helmetless surgical hood and gown support 50, operating condition parameter sensor 602 (and/or 602a in FIG. 4), and microphone assembly 604. In one embodiment, operating condition parameter sensor 602 and/or 602a can include, but is not limited to, a carbon dioxide (CO₂) sensor, a temperature sensor, a humidity sensor, oxygen (O₂) sensor, volatile organic compounds (VOCs) sensor, and/or an air pressure level sensor. The operating condition parameter sensor 602 (and/or 602a) is configured to be used to measure air quality within hood 14 while the hood 14 is being worn by the user. In one embodiment, the air quality can be related to, but not limited to, a carbon dioxide (CO₂) level, a temperature level, a humidity level, an oxygen (O₂) level, a volatile organic compounds (VOCs) level, and/or an air pressure level within the hood 14 while the hood 14 is being worn by the user. It is to be understood that operating condition parameter sensor 602 and/or 602a are configured to continuously send the air quality within the surgical hood data to the printed circuit board 502 (and the processor 902) regarding the operating conditions within the hood 14 and the printed circuit board 502 is configured transmit this information to processor 902. Processor 902 is configured to store this information in storage such as storage 906 and/or transmit the data to the mobile application 967 and/or the cloud computing network 1460 through transceivers 904 and 969 (FIG. 9). It is to be understood that transceivers 904 is configured to interact processor 902 and transceiver 969 is configured to interact with mobile application 967 that is conventionally installed on a remote computer 2065.

As shown in FIG. 7, in one embodiment, the operating condition parameter sensor 602 is connected to the helmetless surgical hood and gown support 50 so that operating condition parameter sensor 602 can be securely located adjacent to the mouth of the wearer. It is to be understood that operating condition parameter measurement assembly 600 can be adjusted so as to be able to position the operating parameter condition measurement assembly 600 within a desired distance away from the wearer's mouth. It is to be further understood that while the operating condition parameter sensor 602 is being used in conjunction with the helmetless surgical hood and gown support 50 and hood 14, operating condition parameter sensor 602 can be used on other medical devices located within the hood 14. Furthermore, operating condition parameter sensor 602 could be used in conjunction with filter 202 to measure the quality of the air that is being introduced into filter 202.

In another embodiment, operating condition parameter measurement assembly 600 can also include an operating condition parameter sensor 602a which can also be located adjacent to the fan motor 250 (FIG. 4) instead of being located adjacent to the user's mouth (FIG. 7). In still another embodiment, both operating condition parameter sensor 602 and 602a can be utilized within hood 14. It is to be understood no matter if either or both operating condition parameter sensor 602 and/or 602a are utilized within hood 14, the operating condition parameter sensor 602 and/or 602a should be located within the hood 14 so that the operating condition parameter sensor 602 and/or 602a can monitor the operating condition parameters or air quality (i.e., CO₂ levels, temperature, humidity levels, oxygen (O₂) levels, volatile organic compounds (VOCs), etc.) within the hood 14 while the hood 14 is being used during a medical procedure.

In another embodiment, the operating condition parameter measurement assembly 600 can also be equipped with a microphone assembly 604. In this manner, the microphone assembly 604 can be used to allow the wearer to communicate with other personnel in the area where the medical procedure is being performed and/or personnel who are observing the medical procedure at a location remote from the medical procedure area.

### Operation of System for Managing Medical Device Maintenance and Medical Device Consumables

With respect to the operation of the system for managing medical device maintenance and medical device consumables, attention is directed to FIGS. 1-9. Assume that a medical device such as a surgical gown 4 (FIG. 1) is equipped with a conventional gown wireless identification system 30 having an RFID tag 32 that can be conventionally attached to or electrically connected to the surgical gown 4, as discussed earlier. Secondly, assume that another medical device such as a fan filter 202 (FIG. 4) is equipped with a conventional wireless identification system 208 having an RFID tag 209 that can be conventionally attached to or electronically connected to the air filter 202. Thirdly, assume that a still another medical device such as a battery 252 (FIGS. 4 and 5) is equipped with a conventional power module wireless identification system 258 having an RFID tag that can be conventionally attached to or electronically connected to the battery 252. Fourthly, assume that a fan motor 260 (FIG. 4) is equipped with a motor sensor/wireless identification system 262 having a sensor such as a tachometer 263 and RFID tag 261 that can be conventionally attached to or electronically connected to the fan motor 260. It is to be understood that a processor 902 (FIG. 14) in conjunction with printed circuit board (PCB) module 500 can use the information from tachometer 263 or any other similar device in the motor sensor/wireless identification system 262 to determine the speed at which the motor 260 is operating. Finally, assume that gown wireless identification system 30, air filtration module wireless identification system 208, power module wireless identification system 258, motor sensor/wireless identification system 262, and the operating condition parameter measurement assembly 600 are in electrical communication with printed circuit board 502 and processor 902.

In another embodiment, information from tachometer 263 or any other similar device in the motor sensor/wireless identification system 262 can be sent through printed circuit board 502 to processor 902 so that processor 902 in conjunction with printed circuit board (PCB) module 500 can control the speed of fan motor 260. In particular, operating condition parameter measurement assembly 600 (i.e., operating condition parameter sensor 602 and/or 602a) can be used to provide information to processor 902 regarding the operating conditions within the hood 14 (FIG. 7). For example, operating condition parameter sensor 602 and/or 602a can be used to detect carbon dioxide (CO₂), temperature, humidity, oxygen (O₂), VOCs, and/or air pressure levels within hood 14. The operating conditions can be monitored while the wearer is donning the hood 14. The operating conditions data from operating conditions parameter sensor 602 and 602a can be transmitted through the printed circuit board (PCB) module 500 (and printed circuit board 502) to the processor 902. In another embodiment, the processor 902 has been configured with operating condition parameter thresholds (such as CO₂ level should not exceed 1,200 ppm or 2,500 ppm depending upon the desired operating conditions). As discussed above, an algorithm running on the printed control board 265 determines the operating speed of the fan motor 260 based on one or more pre-determined thresholds, and the algorithm could be updated with data from the processor 902 (FIG. 9) and uploaded onto the printed control board 265 at a desired interval.

In another embodiment, the tachometer 263 can then be utilized to monitor a “full” condition. For example, tachometer 263 can be used to detect if filter 202 is clogged, motor 260 is failing, or the like. For example, if the tachometer 263 measures a reduced speed of the motor 260, the processor 902 is configured to determine that filter 202 is clogged, motor 260 is failing, or the like.

In another embodiment, as shown in FIG. 9, the operating condition information forwarded from the operating parameter sensor 602 and/or 602a to the processor 902 can also be stored in data storage 906 or forwarded to a mobile application 967 that is configured on a remote computer 2065 associated with the wearer or a system administrator, wherein the mobile application 967 is configured to provide the wearer and/or system administrator with access to the operating conditions within the hood 14 while the wearer is donning the hood 14. For example, the mobile application 967 is configured to display on a display 970 on the remote computer 2065. The operating condition information can then be forwarded from the remote computer 2065 to a cloud computing network 1460 for storage on the cloud computing network 1460. Also, the operating condition information can also be stored on the remote computer 2065 in storage 971.

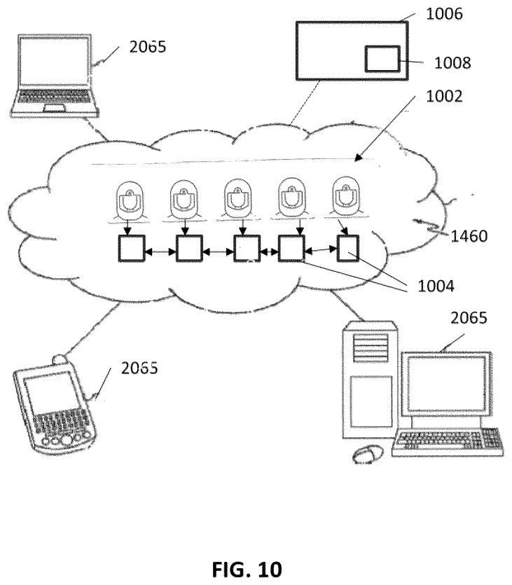

With respect to FIG. 10, another unique aspect of the present invention will now be described. In particular, the cloud computing network 1460 can be used to store some or all of the operating conditions from the operating parameter sensors 602 and/or 602a for each of the wearer/medical personal 1002. Typically, the cloud computing network 906 includes one or more cloud computing nodes 1004 with which local computing devices 2065 used by wearer 1002 may communicate. Nodes 1004 may communicate with one another. They may be grouped (not shown) physically or virtually, in one or more networks, such as Private, Community, Public, or Hybrid clouds as described hereinabove, or a combination thereof. This allows cloud computing network 1460 to offer infrastructure, platforms and/or software as services for which a cloud consumer does not need to maintain resources on a local computing device. Also, the cloud computing network 906 includes a server 1006 having a predictive model 1008, as will be discussed in greater detail later.

With respect to the present invention, medical device users and manufacturers and administrators of medical device systems desire to know how a medical device operates during a medical procedure. For example, if a medical procedure is going to take several hours to complete and the medical personnel 1002 will need to use several different pieces of medical equipment (i.e., a surgical hood 14 having a ventilation system 100 (FIG. 1)) during the medical procedure, it is desirable to be able to continuously monitor the environment within the surgical hood 14 and the operation of the ventilation system 100 to ensure that the medical personnel 1002 will be able to complete the medical procedure during the required time period. It would be further desired if the information related to the monitoring of the environment within the surgical hood 14 and the operation of the ventilation system 100 could be forwarded to a cloud computing network 1460, a processor 902, and/or a mobile application 967 for subsequent analysis of the operating conditions of the environment within the surgical hood 14 and the operation of the ventilation system 100.

Now assume that there are many medical personnel 1002 performing medical procedures at the same time and that these medical personnel 1002 are located in different medical/surgical facilities. Also, assume that these medical personnel 1002 are also wearing a surgical hood 14 having a ventilation system 100. It would be desirable to continuously monitor the environment within the surgical hood 14 and the operation of the ventilation system 100 of all of these medical personnel 1002 and forward the information related to the monitoring of the environment within the surgical hood 14 and the operation of the ventilation system 100 for all of the medical personnel 1002 to a cloud computing network 1460 (FIG. 10), a processor 902, and/or a mobile application 967 for subsequent analysis of the operating conditions of the environment within the surgical hoods 14 and the operation of the ventilation systems 100. In this manner, the system of the present invention will then be able to collect information about the operating conditions of the environment within the surgical hoods 14 and the operation of the ventilation systems 100 from a broad range of data sources (i.e., other medical personnel 1002) in real-time in order to more accurately identify operating conditions that are exceeding a desired threshold, correlate these operating conditions that are exceeding a desired threshold, and provide a recommended solution for correcting the operating conditions that are exceeding a desired threshold.

### Using a Predictive Model

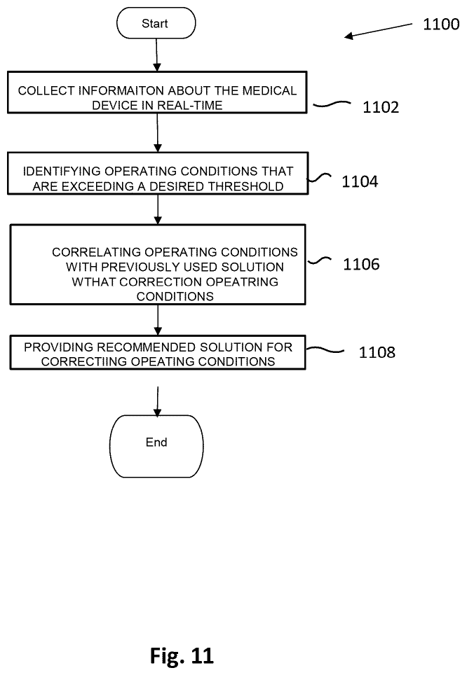

With respect to FIG. 11, there is illustrated a method 1100 for collecting information about the medical device in real-time (step 1102), identifying operating conditions that are exceeding a desired threshold (step 1104), correlating these operating conditions that are exceeding a desired threshold with a previously used solution for correcting the condition that are exceeding a desired threshold (step 1106), and providing a recommended solution for correcting the operating conditions that are exceeding a desired threshold (step 1108). It is to be understood that method 1100 utilizes one or more machine learning models to obtain better predictive performance than could be obtained from any of the constituent learning algorithms alone. It is also to be understood that the machine learning model may be generated using any possible predictive model training operation, such as regression, logistic regression, decision trees, artificial neural networks, support vector machines, linear regression, nearest neighbor methods, distance-based methods, naive Bayes, linear discriminant analysis, k-nearest neighbor algorithm, etc. It is to be understood that the predictive model 1108 can be located on the medical device (i.e., ventilation system 100), the mobile device 2065, and/or the server 1006.

### Training the Predictive Model

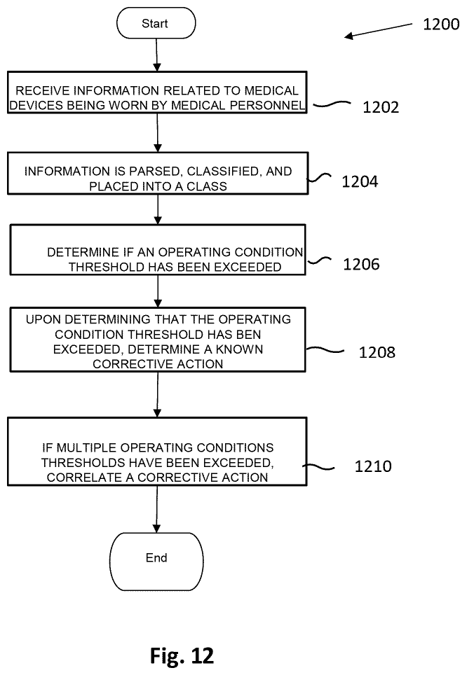

Regarding step 1102, as shown in FIG. 12, there is illustrated a method 1200 for collecting information about the medical device in real-time in order to train a predictive model 1008 located on a server 1006, according to one embodiment. The method 1200 begins at block 1202 with the medical device (i.e., ventilation system 100), the mobile device 2065, and/or server 1006 receiving the information related to the operating conditions of the medical devices being worn by the medical personnel 1002, as discussed earlier with respect to FIG. 10. It is to be understood that this information is referred to as the “known information related to the operating conditions of the medical device (i.e., ventilation system 100).”

At block 1204, the known information is parsed and classified into various classifications by predictive model 1008. For example, a class data set can be created that is related to the operating conditions of a particular medical device (i.e., the ventilation system 100 such as a fan motor 260 located in the ventilation system 100 based upon the data obtained from the sensor (i.e., tachometer 263) and/or battery 252 based upon data obtained from battery sensor 253 forwarded to processor 902, as discussed above). Sub-class data sets can then be created that are related to the operating condition of the particular medical device (the fan motor 260 and/or the battery sensor 253). For example, information related to CO₂ levels within hood 14 while the medical device is being used can be placed into one sub-class. Information related to VOC levels can be placed into another subclass. Information related to temperature within the surgical hood 14 is placed into another class. It is to be understood that a class can be set up for each of the various medical devices being used during a medical procedure. Also, operating conditions sub-classes can be set up for each of the various medical devices being used during a medical procedure.

At block 1206, in order to train the predictive model 1008, predictive model 1008 determines if the known information (including the classified information) is related to an operating condition that exceeded an operating condition parameter threshold. For example, are the CO₂ levels within the surgical hood 14 above a desired threshold? Also, are the VOC levels within the surgical hood 14 above a desired threshold? Finally, is the temperature within the surgical hood 14 above a desired threshold?

At block 1208, the predictive model 1008 is provided by the processor 902 with known corrective actions that have been taken in the past to address (i.e., correct) the operating conditions of the medical device (i.e., the ventilation system 100) that exceeded desired thresholds in the past. It is to be understood that these known corrective actions are stored in a database. For example, if one of the thresholds of an operating condition (carbon dioxide (CO₂), temperature, humidity, oxygen (O₂), VOCs, and/or air pressure levels) is exceeded, a known corrective action may include having the processor 902 in conjunction with printed circuit board (PCB) module 500 interact with the fan motor 260 to adjust the speed of the fan motor 260. In another embodiment, the tachometer 263 can then be utilized to monitor a “full” condition. For example, tachometer 263 can be used to detect if filter 202 is clogged, motor 260 is failing, or the like. In this manner, the predictive model 1008 is trained with a set of known operating conditions that exceeded an operating condition parameter threshold and with the known corrective actions that have been taken in the past to address (i.e., correct) the operating conditions that exceeded an operating condition parameter threshold.

At block 1210, the predictive model 1008 is provided with at least one, current operating conditions that exceed desired thresholds. The predictive model 1008 is then trained to correlate a known corrective action that can be used to address (or correct) the operating conditions that are exceeding desired thresholds. Using the example above, if the predictive model 1008 determines that thresholds of an operating condition (carbon dioxide (CO₂), temperature, and VOCs) are exceeded, the predictive model 1008 can be trained to recommend that the processor 902 in conjunction with printed circuit board (PCB) module 500 interact with the fan motor 260 to adjust the speed of the fan motor 260. In this manner, the predictive model 1008 determines if the current operating condition of the medical device such as ventilation system 100 is exceeded an operating condition parameter threshold that is similar to at least one of the known operating condition parameter thresholds. If the predictive model 1008 does determine that the current operating condition of the medical device such as ventilation system 100 is exceeded an operating condition parameter threshold that is similar to at least one of the known operating condition parameter thresholds, the predictive model 1008 can be trained to query the database for known corrective actions that have been used in the past to correct similar medical device operating condition that exceeded the operating condition parameter threshold. Upon finding known corrective actions, the predictive model 1008 retrieves the known corrective actions from the database. The predictive model 1008 is trained to correlate the known corrective action with the current operating condition of the ventilation system 100 that is exceeded an operating condition parameter threshold to create a recommended corrective action. The predictive model 1008 is then configured to generate an electronic message, wherein the electronic message includes a data structure which includes the recommended corrective action. For example, the predictive model 1008 can recommend (the “recommended corrective action”) that the air filter 202 should be checked to determine if the air filter 202 is clogged. This recommended corrective action can also be in form of an alert, wherein the mobile application 967 is configured receive the recommended corrective action and then display the alert on the display 970.

As discussed above, a unique aspect of the present invention is that the system and method can be configured to monitor and correlate the following operating condition parameters of a medical device:
1. Fan speed input in cubic feet per minute (cfm)
2. Revolutions per minute (RPM) of the fan
3. Air Filters and filter life expectancy
4. CO₂ and O₂ levels
5. Air pressure levels
6. Temperature within the surgical hood and the operating room
7. Humidity within the surgical hood and the operating room
8. Volatile organic compound levels (VOCs)
9. Time since surgery started
10. Battery State of Charge (SOC)
11. Light brightness of any light being used within the surgical hood

For example, known corrective actions related to correcting CO₂, O₂, and VOC levels that are exceeding threshold levels may be to adjust fan speed, replace air filter, and/or replace fan motor. Also, known corrective actions related to temperature and humidity levels that are exceeding threshold levels may be to adjust fan speed, replace air filter, and/or replace fan motor. Also, known corrective actions related to correcting battery SOC levels that are exceeding threshold levels may be to check battery power level, check battery connection; check battery temperature, and replace battery. Finally, known corrective actions related to light brightness levels that are exceeding threshold levels may be to check battery power level, check battery connection; check battery temperature, replace battery, check light, and replace light. In this manner, once the predictive model 1008 is provided with multiple operating conditions that exceed desired thresholds, the predictive model 1008 is configured to provide (or predict) recommended corrective actions in order to correct the multiple operating conditions that are exceeding desired thresholds.

A unique aspect of the present invention is that the predictive model 1008 can also be trained to provide a ranking for recommended corrective actions. For example, if the thresholds of an operating condition (carbon dioxide (CO₂), temperature, and VOCs) is exceeded, the predictive model 1008 can be trained to recommend the following ranked corrective actions:
1. Adjust fan speed.
2. Replace fan filter.
3. Replace fan motor.
   In this manner, the predictive model 1008 would provide a series of corrective actions with the suggested first corrective action being adjust the fan speed, the second corrective action being replace the fan filter, and the third corrective action being replace the fan motor. It is to be understood that the series of corrective actions with the suggested rankings can be in form of an alert, wherein the mobile application 967 is configured receive the series of corrective actions with the suggested rankings can be in form of an alert and then display this alert on the display 970. In response to receiving this alert, the corrective action is completed on the medical device such as the ventilation system 100.

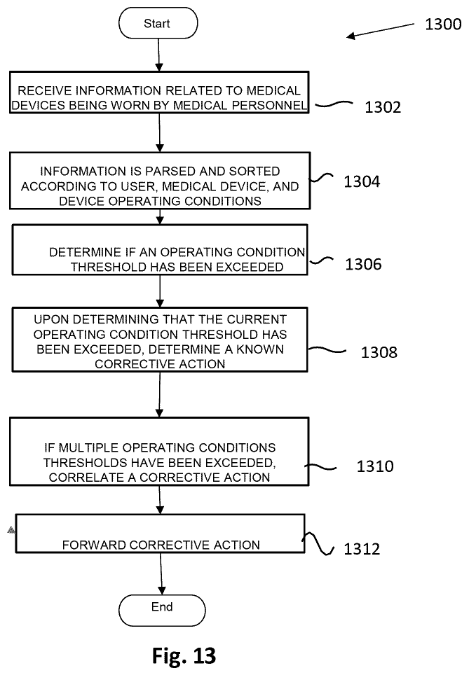

With respect to FIG. 13, there is illustrated a method 1300 for determining if there is a correlation between the various operating condition parameters of a medical device and how the medical device is operating, and provide (or predict) a recommended corrective action to correct the operating conditions of the medical device by using the predictive model 1008, according to one embodiment. The method 1300 begins at block 1302 with the medical device (i.e., ventilation system 100), the mobile device 2065, and/or the server 1006 receiving new information related to the operating conditions of the medical devices being worn by the medical personnel, as discussed earlier with respect to FIG. 10. It is to be understood that the new information is information related to the operating conditions of the medical devices being worn by the medical personnel that were not previously used to train the predictive model 1008.

At block 1304, the new information is parsed and sorted according to the user/medical personnel 1002, the medical device being used (i.e., class), and the operating conditions of the medical device by predictive model 1008 (i.e., sub-class), similarly to the classes/sub-classes, as discussed above. For example, the new information could be parsed, sorted, and saved in a database such as database 1416 under the user/medical personnel 1002 and the medical devices that the medical personnel are currently using and the real-time operating conditions of the medical device. It is to be understood that the database can be configured to include a different set (class) of data for each user/medical personnel and a different sub-set (sub-class) of data for each operating condition for each medical device being used by a user/medical personnel.

At block 1306, predictive model 1008 determines if a medical device's operating condition is exceeded an operating condition parameter threshold. The predictive model 1008 can query known operating condition parameter thresholds stored in the database such as database 1416 and determine if the current medical device operating condition is exceeded an operating condition parameter threshold that is similar to at least one of the known operating condition parameter thresholds.

At block 1308, if the predictive model 1008 determines that the current medical device operating condition is exceeded an operating condition parameter threshold that is similar to at least one of the known operating condition parameter thresholds., the predictive model 1008 can query known corrective actions that have been used to correct similar medical device operating condition that exceeded an operating condition parameter threshold in the past from the database, upon finding known corrective actions in the database, retrieve the known corrective actions from the database, and correlate a known corrective action that is related to correcting the operating condition parameter threshold stored in the database (i.e., the recommended corrective action). The predictive model 1008 can then assign (display) the corrective action to the set of data related to the user/medical personnel and the sub-set of data related to the medical device being used by the user/medical personnel 1002.

At block 1310, if the predictive model 1008 is provided with multiple operating conditions of the medical device that exceed desired thresholds, the predictive model 1008 then correlates a corrective action that can be used to address the multiple operating conditions that are exceeding desired thresholds. The predictive model 1008 can then assign the corrective action to the set of data related to the user/medical personnel and the sub-set of data related to the medical device being used by the user/medical personnel 1002.

At block 1312, the predictive model 1008 then retrieves the corrective action (or actions) from the database 1416 assigned to the user/medical personnel and the medical device being used by the user/medical personnel 1002 and forwards (transmits) the recommended corrective actions to the user and/or a system administrator associated with the medical device through mobile application 967 on a remote computer such as mobile device 2065. In particular, the mobile application 967 is configured to generate an electronic message, wherein the electronic message includes a data structure which includes the recommended corrective action to be reviewed by the user and/or system administrator on display 970. If the user and/or system administrator agree with the recommended corrective action, the corrective action is then taken (or completed) on the medical device. It is to be understood that the corrective action can also be forwarded to the manufacturer of the medical device for user edification and/or further product development.

It is to be understood that in one embodiment, if needed, the mobile application 967 can be configured to set up a video conference to assist the user and/or system administrator in reviewing the recommended corrective action. For example, if the corrective action is to replace a fan motor, a video conference call can be set up between a medical device service technician and the user and/or system administrator. During the video conference call, the service technician could perform further diagnostic evaluations on the fan motor to ensure that the fan motor needs to be replaced. If during the video conference call, it is determined that the fan motor does need to be replaced, the service technician could then set up an appointment with the user and/or system administrator so that the fan motor can be replaced. During the video conference, the mobile application 967 could be connected to the medical device (such as through a Bluetooth® connection) and receiving data from the remote service technician or the predictive model 1008 to analyze to determine the problem. In this manner, the video conference could be conducted through the mobile application 967 running on the mobile device (or another mobile device) 965 while the system is actively collecting and analyzing data to determine the problem.

### Computing Device Embodiment

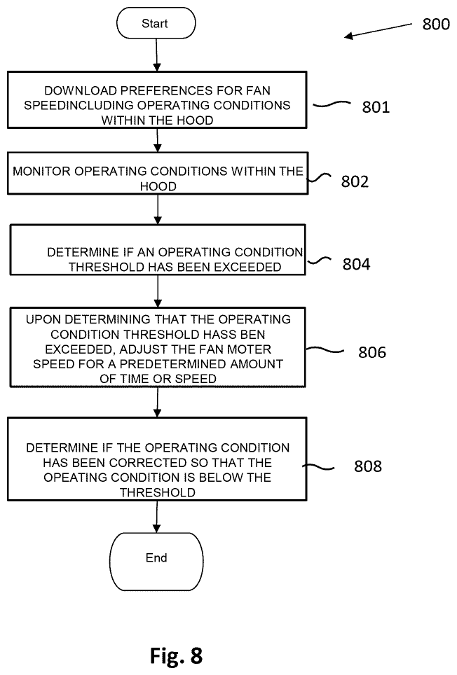

FIG. 14 illustrates an example computing device that is configured and/or programmed as a special purpose computing device with one or more of the example systems and methods described herein, and/or equivalents. The example computing device may be a computer 1400 that includes at least one hardware processor 902, a memory 1404, and input/output ports 1410 operably connected by a bus 1408. In one example, the computer 1400 may include logic 1430 similar to logic/system 800, 1100, 1200, and 1300 shown in FIGS. 8 and 11-13.

In different examples, the logic 1430 may be implemented in hardware, a non-transitory computer-readable medium 1437 with stored instructions, firmware, and/or combinations thereof. While the logic 1430 is illustrated as a hardware component attached to the bus 1408, it is to be appreciated that in other embodiments, the logic 1430 could be implemented in the processor 902, stored in memory 1404, or stored in disk 906.

In one embodiment, logic 1430 or the computer is a means (e.g., structure: hardware, non-transitory computer-readable medium, firmware) for performing the actions described. In some embodiments, the computing device may be a server operating in a cloud computing system, a server configured in a Software as a Service (SaaS) architecture, a smart phone, laptop, tablet computing device, and so on.

The means may be implemented, for example, as an ASIC programmed to predict a product demand. The means may also be implemented as stored computer executable instructions that are presented to computer 1400 as data 1416 that are temporarily stored in memory 1404 and then executed by processor 902.

Logic 1430 may also provide means (e.g., hardware, non-transitory computer-readable medium that stores executable instructions, firmware) for storing and measuring operating condition parameters.

Generally describing an example configuration of the computer 1400, the processor 902 may be a variety of various processors including dual microprocessor and other multi-processor architectures. A memory 1404 may include volatile memory and/or non-volatile memory. Non-volatile memory may include, for example, ROM, PROM, and so on. Volatile memory may include, for example, RAM, SRAM, DRAM, and so on.

A storage disk 906 may be operably connected to the computer 1400 via, for example, an input/output (I/O) interface (e.g., card, device) 1418 and an input/output port 1410 that are controlled by at least an input/output (I/O) controller 1440. The disk 906 may be, for example, a magnetic disk drive, a solid-state disk drive, a floppy disk drive, a tape drive, a Zip drive, a flash memory card, a memory stick, and so on. Furthermore, the disk 906 may be a CD-ROM drive, a CD-R drive, a CD-RW drive, a DVD ROM, and so on. The memory 1404 can store a process 1414 and/or a data 1416, for example. The disk 906 and/or the memory 1404 can store an operating system that controls and allocates resources of the computer 1400.

The computer 1400 may interact with, control, and/or be controlled by input/output (I/O) devices via the input/output (I/O) controller 1440, the I/O interfaces 1418, and the input/output ports 1410. Input/output devices may include, for example, one or more displays 1470, printers 1472 (such as inkjet, laser, or 3D printers), audio output devices 1474 (such as speakers or headphones), text input devices 1480 (such as keyboards), cursor control devices 1482 for pointing and selection inputs (such as mice, trackballs, touch screens, joysticks, pointing sticks, electronic styluses, electronic pen tablets), audio input devices 1484 (such as microphones or external audio players), video input devices 1486 (such as video and still cameras, or external video players), image scanners 1488, video cards (not shown), disks 906, network devices 1420, and so on. The input/output ports 1410 may include, for example, serial ports, parallel ports, and USB ports.

The computer 1400 can operate in a network environment and thus may be connected to the network devices 1420 via the I/O interfaces 1418, and/or the I/O ports 1410. Through the network devices 1420, the computer 1400 may interact with a network 1460. Through the network, the computer 1400 may be logically connected to remote computers 2065. Networks with which the computer 1400 may interact include, but are not limited to, a LAN, a WAN, and other networks.

### Definitions and Other Embodiments

In another embodiment, the described methods and/or their equivalents may be implemented with computer executable instructions. Thus, in one embodiment, a non-transitory computer readable/storage medium is configured with stored computer executable instructions of an algorithm/executable application that when executed by a machine(s) cause the machine(s) (and/or associated components) to perform the method. Example machines include but are not limited to a processor, a computer, a server operating in a cloud computing system, a server configured in a Software as a Service (SaaS) architecture, a smart phone, and so on). In one embodiment, a computing device is implemented with one or more executable algorithms that are configured to perform any of the disclosed methods.

In one or more embodiments, the disclosed methods or their equivalents are performed by either: computer hardware configured to perform the method; or computer instructions embodied in a module stored in a non-transitory computer-readable medium where the instructions are configured as an executable algorithm configured to perform the method when executed by at least a processor of a computing device.

While for purposes of simplicity of explanation, the illustrated methodologies in the figures are shown and described as a series of blocks of an algorithm, it is to be appreciated that the methodologies are not limited by the order of the blocks. Some blocks can occur in different orders and/or concurrently with other blocks from that shown and described. Moreover, less than all the illustrated blocks may be used to implement an example methodology. Blocks may be combined or separated into multiple actions/components. Furthermore, additional and/or alternative methodologies can employ additional actions that are not illustrated in blocks. The methods described herein are limited to statutory subject matter under 35 U.S.C § 101.

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

A “data structure”, as used herein, is an organization of data in a computing system that is stored in a memory, a storage device, or other computerized system. A data structure may be any one of, for example, a data field, a data file, a data array, a data record, a database, a data table, a graph, a tree, a linked list, and so on. A data structure may be formed from and contain many other data structures (e.g., a database includes many data records). Other examples of data structures are possible as well, in accordance with other embodiments.

“Computer-readable medium” or “computer storage medium”, as used herein, refers to a non-transitory medium that stores instructions and/or data configured to perform one or more of the disclosed functions when executed. Data may function as instructions in some embodiments. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an application specific integrated circuit (ASIC), a programmable logic device, a compact disk (CD), other optical medium, a random access memory (RAM), a read only memory (ROM), a memory chip or card, a memory stick, solid state storage device (SSD), flash drive, and other media from which a computer, a processor or other electronic device can function with. Each type of media, if selected for implementation in one embodiment, may include stored instructions of an algorithm configured to perform one or more of the disclosed and/or claimed functions. Computer-readable media described herein are limited to statutory subject matter under 35 U.S.C § 101.

“Logic”, as used herein, represents a component that is implemented with computer or electrical hardware, a non-transitory medium with stored instructions of an executable application or program module, and/or combinations of these to perform any of the functions or actions as disclosed herein, and/or to cause a function or action from another logic, method, and/or system to be performed as disclosed herein. Equivalent logic may include firmware, a microprocessor programmed with an algorithm, a discrete logic (e.g., ASIC), at least one circuit, an analog circuit, a digital circuit, a programmed logic device, a memory device containing instructions of an algorithm, and so on, any of which may be configured to perform one or more of the disclosed functions. In one embodiment, logic may include one or more gates, combinations of gates, or other circuit components configured to perform one or more of the disclosed functions. Where multiple logics are described, it may be possible to incorporate the multiple logics into one logic. Similarly, where a single logic is described, it may be possible to distribute that single logic between multiple logics. In one embodiment, one or more of these logics are corresponding structure associated with performing the disclosed and/or claimed functions. Choice of which type of logic to implement may be based on desired system conditions or specifications. For example, if greater speed is a consideration, then hardware would be selected to implement functions. If a lower cost is a consideration, then stored instructions/executable application would be selected to implement the functions. Logic is limited to statutory subject matter under 35 U.S.C. § 101.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, non-transitory computer-readable medium). Logical and/or physical communication channels can be used to create an operable connection.

“User”, as used herein, includes but is not limited to one or more persons, computers or other devices, or combinations of these.

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or the illustrative examples shown and described. Thus, this disclosure is intended to embrace alterations, modifications, and variations that fall within the scope of the appended claims, which satisfy the statutory subject matter requirements of 35 U.S.C. § 101.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

Therefore, provided herein is a new and improved system and method for managing medical devices and medical device consumables, which according to various embodiments of the present invention, offers the following advantages: ease of use; the ability to keep track of operating condition parameters in medical devices; the ability to train the system and method to be able to correlate various operating condition parameters with previous recommendations on how to correct the operating condition parameter in the medical device; the ability to the ability to provided recommendations on how to correct the operating condition parameter in the medical device without user intervention; and the ability to provide feedback regarding the operating condition parameters in the medical device for wearer edification, preventative maintenance, and/or further product development.

In fact, in many of the preferred embodiments, these advantages of ease of use, the ability to keep track of operating condition parameters in medical devices, the ability to train the system and method to be able to correlate various operating condition parameters with previous recommendations on how to correct the operating condition parameter in the medical device, the ability to the ability to provided recommendations on how to correct the operating condition parameter in the medical device without user intervention, and the ability to provide feedback regarding the operating condition parameters in the medical device for wearer edification, preventative maintenance, and/or further product development are optimized to an extent that is considerably higher than heretofore achieved in prior, known systems and methods for managing operating condition parameters in medical devices.

What is claimed is:

1. A computer-implemented method for predicting a recommended corrective action to correct an operating condition of a medical device performed by a computing device, where the computing device includes at least a processor for executing instructions from a memory, the method comprising:
providing a surgical gown;
providing a surgical hood operatively connected to the surgical gown, wherein the hood is configured to be located over a head and neck area of a wearer such that the head and neck area of the wearer are substantially enclosed within the hood;
providing a medical device located within the surgical gown and the surgical hood, wherein the medical device includes a ventilation system that is configured to be retained by shoulders of the wearer of the ventilation system in order to provide ventilation air within the surgical gown and surgical hood;
training, using a machine learning model, a recommended corrective action predictor associated with correcting an operating condition of the ventilation system, wherein the recommended corrective action predictor implements machine learning techniques comprising a neural network, wherein the recommended corrective action predictor was trained using data obtained from a sensor associated with the ventilation system, wherein the data is related to known operating conditions of the ventilation system; and wherein the data related to known operating conditions of the ventilation system is stored in a database;
determining, by the recommended corrective action predictor, if the data related to known operating conditions of the ventilation system is related to a known operating condition that exceeded an operating condition parameter threshold;
providing, from the processor to the recommended corrective action predictor, known corrective actions that have previously been used to correct known operating conditions of the ventilation system that exceeded desired thresholds, wherein the known corrective actions are stored in a database;
providing, to the recommended corrective action predictor, at least one current operating condition of the ventilation system;
determining, by the recommended corrective action predictor, if the at least one current operating condition of the ventilation system is exceeding an operating condition parameter threshold;
determining, by the recommended corrective action predictor, if the current operating condition of the ventilation system is exceeded an operating condition parameter threshold that is similar to at least one of the known operating condition parameter thresholds;
upon determining, by the recommended corrective action predictor, that the current operating condition of the ventilation system is exceeded an operating condition parameter threshold that is similar to at least one of the known operating condition parameter thresholds, querying, by the recommended corrective action predictor, the database for known corrective actions that have been used to correct a similar medical device operating condition that exceeded the operating condition parameter threshold;
upon finding known corrective actions, by the recommended corrective action predictor, retrieving the known corrective actions from the database;
correlating, by the recommended corrective action predictor, the known corrective action with the current operating condition of the ventilation system that is exceeded an operating condition parameter threshold to create a recommended corrective action;
generating, by the recommended corrective action predictor, an electronic message, wherein the electronic message includes a data structure which includes the recommended corrective action;
transmitting, by the recommended corrective action predictor, the electronic message to a remote computer associated with the a user; and
in response to receiving the electronic message, completing the corrective action on the ventilation system.

2. The method of claim 1, wherein the method further comprises:
parsing, by the recommended corrective action predictor, the data related to known operating conditions of the ventilation system; and
classifying, by the recommended corrective action predictor, the data related to known operating conditions of the ventilation system.

3. The method of claim 1, wherein the sensor further comprises:
a battery sensor.

4. The method of claim 1, wherein the sensor further comprises:
a tachometer.

5. The method of claim 3, wherein the battery sensor is configured to monitor an operating status of a battery to ensure that the battery is operating properly and to send data to the processor regarding the operating status of the battery.

6. The method of claim 4, wherein the tachometer is configured to monitor an operating speed of a fan motor and continuously send data to the processor regarding the operating speed of the fan motor.

7. The method of claim 1, wherein the sensor further comprises:
an operating condition parameter sensor, wherein the operating condition parameter sensor is configured to measure air quality within the surgical hood and is configured to continuously send the air quality within the surgical hood data to the processor.

8. A non-transitory computer-readable medium storing computer-executable instructions that, when executed by a computer including a processor, cause the computer to perform functions configured by the computer-executable instructions for predicting a recommended corrective action to correct an operating condition of a medical device, wherein the instructions comprise:
providing a surgical gown;
providing a surgical hood operatively connected to the surgical gown, wherein the hood is configured to be located over a head and neck area of a wearer such that the head and neck area of the wearer are substantially enclosed within the hood;
providing a medical device located within the surgical gown and the surgical hood, wherein the medical device includes a ventilation system that is configured to be retained by shoulders of the wearer of the ventilation system in order to provide ventilation air within the surgical gown and surgical hood;
training, using a machine learning model, a recommended corrective action predictor associated with correcting an operating condition of the ventilation system, wherein the recommended corrective action predictor implements machine learning techniques comprising a neural network, wherein the recommended corrective action predictor was trained using data obtained from a sensor associated with the ventilation system, wherein the data is related to known operating conditions of the ventilation system; and wherein the data related to known operating conditions of the ventilation system is stored in a database;
determining, by the recommended corrective action predictor, if the data related to known operating conditions of the ventilation system is related to a known operating condition that exceeded an operating condition parameter threshold;
providing, from the processor to the recommended corrective action predictor, known corrective actions that have previously been used to correct known operating conditions of the ventilation system that exceeded desired thresholds, wherein the known corrective actions are stored in a database;
providing, to the recommended corrective action predictor, at least one current operating condition of the ventilation system;
determining, by the recommended corrective action predictor, if the at least one current operating condition of the ventilation system is exceeding an operating condition parameter threshold;
determining, by the recommended corrective action predictor, if the current operating condition of the ventilation system is exceeded an operating condition parameter threshold that is similar to at least one of the known operating condition parameter thresholds;
upon determining, by the recommended corrective action predictor, that the current operating condition of the ventilation system is exceeded an operating condition parameter threshold that is similar to at least one of the known operating condition parameter thresholds, querying, by the recommended corrective action predictor, the database for known corrective actions that have been used to correct a similar medical device operating condition that exceeded the operating condition parameter threshold;
upon finding known corrective actions, by the recommended corrective action predictor, retrieving the known corrective actions from the database;
correlating, by the recommended corrective action predictor, the known corrective action with the current operating condition of the ventilation system that is exceeded an operating condition parameter threshold to create a recommended corrective action;
generating, by the recommended corrective action predictor, an electronic message, wherein the electronic message includes a data structure which includes the recommended corrective action;
transmitting, by the recommended corrective action predictor, the electronic message to a remote computer associated with the a user; and
in response to receiving the electronic message, completing the corrective action on the ventilation system.

9. The non-transitory computer-readable medium of claim 8, further comprising instructions that, when executed by at least the processor, cause the processor to:
parse, by the recommended corrective action predictor, the data related to known operating conditions of the ventilation system; and
classify, by the recommended corrective action predictor, the data related to known operating conditions of the ventilation system.

10. The non-transitory computer-readable medium of claim 8, wherein the sensor further comprises:
a battery sensor.

11. The non-transitory computer-readable medium of claim 8, wherein the sensor further comprises:
a tachometer, wherein the tachometer is configured to monitor an operating speed of a fan motor and continuously send data to the processor regarding the operating speed of the fan motor.

12. The non-transitory computer-readable medium of claim 10, wherein the battery sensor is configured to monitor an operating status of a battery to ensure that the battery is operating properly and to send data to the processor regarding the operating status of the battery.

13. The non-transitory computer-readable medium of claim 8, wherein the sensor further comprises:
an operating condition parameter sensor, wherein the operating condition parameter sensor is configured to measure air quality within the surgical hood and is configured to continuously send the air quality within the surgical hood data to the processor.

14. A system for predicting a recommended corrective action to correct an operating condition of a medical device, comprising:
a surgical gown;
a surgical hood operatively connected to the surgical gown, wherein the hood is configured to be located over a head and neck area of a wearer such that the head and neck area of the wearer are substantially enclosed within the hood;
a medical device located within the surgical gown and the surgical hood, wherein the medical device includes a ventilation system that is configured to be retained by shoulders of the wearer of the ventilation system in order to provide ventilation air within the surgical gown and surgical hood;
at least one processor connected to at least one memory; and
a non-transitory computer readable medium including instructions stored thereon that, when executed by the at least one processor, cause the at least one processor to:
train, using a machine learning model, a recommended corrective action predictor associated with correcting an operating condition of the ventilation system, wherein the recommended corrective action predictor implements machine learning techniques comprising a neural network, wherein the recommended corrective action predictor was trained using data obtained from a sensor associated with the ventilation system, wherein the data is related to known operating conditions of the ventilation system; and wherein the data related to known operating conditions of the ventilation system is stored in a database;
determine, by the recommended corrective action predictor, if the data related to known operating conditions of the ventilation system is related to a known operating condition that exceeded an operating condition parameter threshold;
provide, from the processor to the recommended corrective action predictor, known corrective actions that have previously been used to correct known operating conditions of the ventilation system that exceeded desired thresholds, wherein the known corrective actions are stored in a database;
provide, to the recommended corrective action predictor, at least one current operating condition of the ventilation system;
determine, by the recommended corrective action predictor, if the at least one current operating condition of the ventilation system is exceeding an operating condition parameter threshold;
determine, by the recommended corrective action predictor, if the current operating condition of the ventilation system is exceeded an operating condition parameter threshold that is similar to at least one of the known operating condition parameter thresholds;
upon determining, by the recommended corrective action predictor, that the current operating condition of the ventilation system is exceeded an operating condition parameter threshold that is similar to at least one of the known operating condition parameter thresholds, query, by the recommended corrective action predictor, the database for known corrective actions that have been used to correct a similar medical device operating condition that exceeded the operating condition parameter threshold;
upon finding known corrective actions, by the recommended corrective action predictor, retrieve the known corrective actions from the database;
correlate, by the recommended corrective action predictor, the known corrective action with the current operating condition of the ventilation system that is exceeded an operating condition parameter threshold to create a recommended corrective action;
generate, by the recommended corrective action predictor, an electronic message, wherein the electronic message includes a data structure which includes the recommended corrective action;
transmit, by the recommended corrective action predictor, the electronic message to a remote computer associated with the a user; and
in response to receiving the electronic message, complete the corrective action on the ventilation system.

15. The system of claim 14, wherein the instructions further include instructions that, when executed by at least the processor, cause the processor to system further comprises:
parse, by the recommended corrective action predictor, the data related to known operating conditions of the ventilation system; and
classify, by the recommended corrective action predictor, the data related to known operating conditions of the ventilation system.

16. The system of claim 14, wherein the sensor further comprises:
a battery sensor.

17. The system of claim 14, wherein the sensor further comprises:
a tachometer.

18. The system of claim 16, wherein the battery sensor is configured to monitor an operating status of a battery to ensure that the battery is operating properly and to send data to the processor regarding the operating status of the battery.

19. The system of claim 17, wherein the tachometer is configured to monitor an operating speed of a fan motor and continuously send data to the processor regarding the operating speed of the fan motor.

20. The system of claim 14, wherein the sensor further comprises:
an operating condition parameter sensor, wherein the operating condition parameter sensor is configured to measure air quality within the surgical hood and is configured to continuously send the air quality within the surgical hood data to the processor.
